(19) United States
(12) Patent Application Publication
(10) Pub. No.: US 2005/0123593 A1
(43) Pub. Date: Jun. 9, 2005

(54) Liposomal encapsulation of glycosaminoglycans for the treatment of arthritic joints

(76) Inventors: Jonathan Thompson, Leeds (GB); Susan Niemiec, Ann Harbor, MI (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/481,605

(22) PCT Filed: Jun. 20, 2002
(86) PCT No.: PCT/US02/19716

Related U.S. Application Data
(60) Provisional application No. 60/386,791, filed on Jun. 7, 2002.
(60) Provisional application No. 60/300,750, filed on Jun. 25, 2001.

(51) Int. Cl. A61K 31/728; A61K 31/727; A61K 9/127

(52) U.S. Cl. 424/450; 514/54; 514/56

(57) ABSTRACT

In a preferred embodiemnt the present invention features a composition and method of delivery comprising Glycosaminoglycans encapsulated in a liposomal delivery system for intraarticular administration for the treatment of osteoarthritis In a more preferred embodiemnt the present invention features a composition and method of delivery comprising hyaluronic acid encapsulated in a liposomal delivery system for intraarticular administration for the treatment of osteoarthritis.

### FIELD OF THE INVENTION

The invention broadly relates to a composition and method for the treatment of arthritic joints.

More specifically, the present invention relates to a composition and method of treatment comprising at least one glycosaminoglycan encapsulated by at least one liposomal system useful for the treatment of arthritic joints.

### BACKGROUND OF THE INVENTION

Glycosaminoglycans (GAGS) are biopolymers consisting of repeating polysaccharide units, and are present on the cell surface as well as in the extracellular matrix of animals. GAGS are long unbranched polysaccharides containing a repeating disaccharide unit. The disaccharide units contain either of two modified sugars, N-acetylgalactosamine or N-acetylglucosamine and a uronic acid such as glucuronate or iduronate. GAGS are highly negatively charged molecules, with extended conformation that imparts high viscosity to the solution. GAGS are located primarily on the surface of cells or in the extracellular matrix. Along with the high viscosity of GAGS comes low compressibility, which makes these molecules ideal for a lubricating fluids in the joints. At the same time, their rigidity provides structural integrity to cells and provides passageways between cells allowing for cell migration.

Common naturally occurring GAGs include, but are not limited to, chondroitin sulphate, keratan sulphate, heparin, heparan sulphate, dermatan sulphate and hyaluronate (commonly referred to as hyaluronic acid, HA). GAGs play an important role in articulating joints, being constituents both of synovial fluid, and of the surface layers of articular cartilage when covalently linked with proteins to form proteoglycans.

Hyaluronic acid (HA) is a high molecular weight polysaccharide of N-acetyl glucosamine and glucuronic acid molecules that is naturally occurring in all mammals in a variety of tissues and some bacterial species. HA is unique among the GAGS in that it does not contain any sulphate and is not found covalently attached to proteins as a proteoglycan. HA polymers are very large with molecular weights of between about 100,000-10,000,000, and can displace a large volume of water.

The chemical structure of hyaluronic acid is:

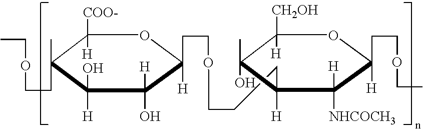

The highest concentrations are found in connective tissue such as synovial membrane and synovial fluid. Hyaluronic acid forms highly viscoelastic solutions, and is synthesized in the plasma membrane of fibroblasts and other cells by addition of sugars to the reducing end of the polymer, whereas the nonreducing end protrudes into the pericellular space. The polysaccharide is catabolized locally or carried by lymph to lymph nodes or the general circulation, from where it is cleared by the endothelial cells of the liver sinusoids. The overall turnover rate is surprisingly rapid for a connective tissue matrix component (t_{1/2}=0.5 to a few days). Methods to prepare pure samples, which are non-inflammatory, are well known in the art. For example, EP 0239335, U.S. Pat. No. 4,879,375, U.S. Pat. No. 4,141,973 disclose methods to prepare highly pure fractions of hyaluronic acid, which purport to be non-inflammatory.

Hyaluronic acid is critical for the homeostasis of the joint, in part, because it provides the rheological properties (viscosity and elasticity) of the synovial fluid. It contributes to joint lubrication, buffers load transmission across articular surfaces, provides a renewed source of HA to joint tissues, and imparts anti-inflammatory properties to synovial fluid. In osteoarthritis, the molecular weight and concentration of HA in synovial fluid are diminished and this impairs the ability of synovial fluid to function effectively. The above observations have led to the development of viscosupplementation by means of intra-articular injections of hyaluronic acid for treatment of osteoarthritis of the knee. This treatment involves removal of pathologic osteoarthritic synovial fluid and replacement with HA-based products that restore the molecular weight and concentration of HA toward normal levels that can have beneficial therapeutic effects. Scientific publications describing the use of hyaluronic acid for treatment of articular conditions are well known in the art examples of which are Adams, 1993, 1996; Adams et al., 1995; Baker, 1997; Balazs, 1968, 1982; Balazs & Denlinger, 1985, 1989, 1993; Balazs & Gibbs, 1970; Band et al., 1995; Denlinger, 1982, 1996; Dickson &Hosle,1998; Estey, 1998; Gibbs et al., 1968; Moreland et al., 1993; Peyron, 1993a,1993b,1999; Rydell et al., 1970; Scale et al., 1994; Weiss et al., 1981; and Weiss & Balaz 1987. In the patent literature hyaluronic acid preparations for treatment of arthritic joints have been described. Examples of which are U.S. Pat. No. 5,914,322, described in U.S. Pat. No. 4,801,619.

Several preparations of HA, e.g. Hyalgan (Fidia S.p.A) and Synvisc (Biomatrix, Inc.), are commercially available as treatments applied via intra-articular injection into the diseased joint. Such treatments have been found to provide significant pain relief, e.g. Peyron and Balazs, 1974; Adams 1993; Adams et al 1995;;; Huskisson and Donnelly 1999, Kotz and Kolarz 1999, by supplementing the synovial fluid with HA which is chemically and mechanically more closely representative of the HA found in young, healthy articular joints.

Although the use of such treatments were reported as early as 1974 [1], the mechanism of action remains poorly understood. While evidence supports several roles of HA within the joint such as viscosupplementation and lubrication, Balazs and Denlinger 1984, protection of the cartilage surfaces, Balazs and Denlinger 1984, and suppression of pain-stimulating mediators such as IL-1α, Balazs and Darrzynkiewicz 1973; Forrester and Balzas 1980; Darzynkiewicz and Balazs, 1971, it is also known that HA molecules are removed from the joint over time through the process of enzymatic breakdown and lymphatic clearance [FDA PMA-Hyalgan 1997; FDA PMA-Hylan G-F 20, 1997; Levick et al 2000. Therefore the longer-term effects of such treatments are limited. Attempts to increase the residence time of HA within the joint have largely focused on modifying the HA molecule by cross-linking FDA PMA-Hylan G-F 2, 1997 [U.S. Pat. No. 5,827,937, WO99/10385], and while this delays clearance of HA there is little evidence to suggest that any additional long-term benefits are derived from such treatments, and concerns remain associated with the altering of the molecular structure, and in some cases the presence of chemical cross-linking agents.

Lipids are also present in joint synovial fluid, and certain phospholipids (in particular dipalmitoylphosphatidylcholine (DPPC)) have been implicated in the lubrication of cartilage surfaces Hills 1995, 2000; Hills and Monds, 1998, and shown to reduce osteoarthritic pain by intra-articular injection into the knee joint Vecchio et al 1999.

Liposomes were first described in 1965 by Bangham (Bangham, A. D., Standish, M. M. and Watkins, J. C. 1965. “Diffusion of Univalent Ions across the lamellae of swollen phospholipid,” J. Mol. Biol., 13: 238-252). Liposomes are classified by size, number of bilayers and hydrophobicity of the core. A conventional liposome is composed of lipid bilayers surrounding a hydrophilic core. The lipids of the lipid bilayers can have conjugating groups such as proteins, antibody polymers, and cationic polyelectrolytes on the surface of the liposomes and will act as targeting surface agents. Lipid vesicles are often classified into three groups by size and structure; multilamellar vesicles (MLVs), large unilamellar vesicles (LUVs), small unilamellar vesicles (SUVs), and paucilamellar (PLVs) vesicles. MLVs are onion-like structures having a series of substantially spherical shells formed of lipid bilayers interspersed with aqueous layers. LUVs have a diameter greater than 1 μm and are formed of a single lipid bilayer surrounding a large hydrophilic core phase. SUVs are similar in structure to LUVs except their diameter is less than an LUV, e.g., less than 100 nm. PLVs are vesicles that have an internal hydrophobic core surrounded by bilayers. See, e.g., Callow and McGrath, Cryobiology, 1985 22(3) pp. 251-267.

Liposomes were initially used as models for studying biological membranes. However, in the last 15 years liposomal delivery systems have been designed as advanced delivery vehicles of drugs and other benefits agents into biological tissues. See, e.g., Gregoriadis, G., ed. 1988. Liposomes as Drug Carriers, New York: John Wiley, pp. 3-18).

Traditionally, the thin-film method was used to manufacture liposomes. In this method, the bilayer-forming elements are mixed with a volatile organic solvent (such as chloroform, ether, ethanol, or a combination of these) in a mixing vessel (such as a round bottom flask). The predominant bilayer-forming element used to form conventional phospholipid vesicles is usually a neutral phospholipid such as phosphatidylcholine. Cholesterol is also often included to provide greater stability of the liposome in biological fluids. A charged species such as phosphatidylserine may also be added to prevent aggregation, and other elements such as natural acidic lipids and antioxidants, may also be included.

The lipid-solvent solution is then placed under specified surrounding conditions (e.g., pressure and temperature) such that the volatile solvent is removed by evaporation (e.g., using a rotary evaporator) resulting in the formation of a dry lipid film. This film is then hydrated with aqueous medium containing dissolved solutes, including buffers, salts, and hydrophilic compounds, that are to be entrapped in the lipid vesicles. The hydration steps used influence the type of liposomes formed (e.g., the number of bilayers formed, vesicle size, and entrapment volume). If desirable, non-encapsulated drug or active can be removed from the mixture by a variety of techniques such as centrifugation, dialysis or diafiltration and recovered.

Combinations of lipids and HA have been variously referenced in the literature. WO-A-91/12026 patented the combination of HA and phospholipid for the treatment of rheumatic joints. It was postulated that by combining HA and DPPC, both of which provide joint lubrication, improved lubrication could be imparted to the cartilage surfaces. A mixture of DPPC liposomes and HA has been shown to remove reduce surgical adhesions post-operatively. In both of these cases the lipid component and the HA component are combined in mixture; therefore no effect on the residence time of the HA molecules would be expected.

Chemical interactions between lipids and GAGs have been described which show hexagonal shaped structures [18,19] or display acid amide bonding between the two ingredients (Aoki et. Al., U.S. Pat. No. 5,470,578, Antirheumatic Composition).

Buttle et. Al. (WO 00/74662 A2, Arthritis Treatment) showed that catechins could be beneficial in the treatment of osteoarthritis and proposed their combination with HA. A liposomal delivery vehicle was mentioned for such a treatment; however, the method for achieving this was unclear as liposomes are generally less than 200 nm in diameter, while the diameter of HA molecules is typically around 200-300 nm [6].

The above prior art does not address the issue of insufficient residence time of HA in vivo. The present invention does address the issue by encapsulating GAG molecules within a liposome, such that these molecules were released over an appropriate time period to provide a treatment with longer-term effects.

Accordingly, the object of the present invention is directed to novel compositions of liposomes and GAGs, which specifically include a liposome of sufficient size to encapsulate the GAG molecules.

### SUMMARY OF THE INVENTION

In a preferred embodiement the present invention features a composition and method of delivery comprising Glycosaminoglycans encapsulated in a liposomal delivery system for intraarticular administration for the treatment of osteoarthritis.

In a more preferred embodiemnt the present invention features a composition and method of delivery comprising hyaluronic acid encapsulated in a liposomal delivery system for intraarticular administration for the treatment of osteoarthritis.

### DETAILED DESCRIPTION OF THE INVENTION

It is believed that one skilled in the art can, based upon the description herein, utilize the present invention to its fullest extent. The following specific embodiments are to be construed as merely illustrative, and not limitative of the remainder of the disclosure in any way whatsoever.

Unless defined otherwise, all technical and scientific terms used herein have the same meaning as commonly understood by one of ordinary skill in the art to which the invention belongs. Also, all publications, patent applications, patents, and other references mentioned herein are incorporated by reference. Unless defined otherwise, all technical and scientific terms used herein have the same meaning as commonly understood by one of ordinary skill in the art to which the invention belongs. Also, all publications, patent applications, patents, and other references mentioned thoroughout this application are herein expressly incorporated by reference.

Glycosaminoglycans (GAGS) are defined as biopolymers consisting of repeating polysaccharide units, and are present on the cell surface as well as in the extracellular matrix of animals. GAGS are long unbranched polysaccharides containing a repeating disaccharide unit. The disaccharide units contain either of two modified sugars, N-acetylgalactosamine or N-acetylglucosamine and a uronic acid such as glucuronate or iduronate. GAGS are highly negatively charged molecules, with extended conformation that imparts high viscosity to the solution. GAGS are located primarily on the surface of cells or in the extracellular matrix. Along with the high viscosity of GAGS comes low compressibility, which makes these molecules ideal for a lubricating fluids in the joints. At the same time, their rigidity provides structural integrity to cells and provides passageways between cells allowing for cell migration.

Hyaluronic acid (HA) is defined as a high molecular weight polysaccharide of N-acetyl glucosamine and glucuronic acid molecules that is naturally occurring in all mammals in a variety of tissue and some bacterial species. For purposes of this applications HA includes any derivatives such as hyaluronan and Hyaluronic acid itself with H+ ion attached to the COO— group. And salts of hyaluronic acid whereby another positive ion replaces the H+ ion, as for example with NA+ which forms sodium hyaluronate. Also included in the definition is any physically or chemically cross-linked hyaluronic acid and deriviatives. HA is unique among the GAGS in that it does not contain any sulphate and is not found covalently attached to proteins as a proteoglycan. HA polymers are very large with molecular weights of between about 100,000-10,000,000, and can displace a large volume of water. For purposes of this invention a most preferred embodiment includes a non cross-linked hyaluronic acid with a molecular weight of 0.5-10 Mda.

Liposomes are defined as small spheres whose walls are layers of lipids with water. As they form, liposomes entrap water and any water soluble solutes that are present. Because of this entrapping ability, they are useful as drug delivery systems. For purposes of the present invention a most preferred embodiment includes the use of a multilamellar vesicle. For purposes of this invention a preferred embodiment includes any naturally occurring phospholipid and a most preferred embodiment includes dipalmitoylphosphatidylcholine (DPPC).

Intra-articular delivery is defined as a method whereby a treatment is delivered, directly or indirectly, into the synovial capsule of an articulating joint.

What is meant by a liposome is a vesicle having at least one lipid bilayer surrounding an inner liquid phase (e.g., either a lipid bilayer surrounding either a liquid core or a liquid phase dispersed between it and another lipid bilayers). The liposome may have various structures such as multilamellar (MLVs), unilamellar (LUVs or SUVs), and paucilamellar (PLVs) as discussed above. The resulting structure of the liposome is dependent, in part, on the choice of materials forming the hydrophobic phase and the manufacturing parameters such temperature and incubation times.

Liposomes manufactured according to the present invention comprise at least one amphiphilic bilayer-forming substance and may comprise a benefit agent.

The benefit agent may be contained either within the lipid bilayer or the hydrophilic or hydrophilic compartments of the liposome.

What is meant by amphiphilic bilayer-forming substance is a lipid that is comprised of both a hydrophilic and lipophilic group and is capable of forming, either alone or in combination with other lipids, the bilayer of a liposome. The lipid can have single or multiple lipophilic side chains being either saturated or unsaturated in nature and branched or linear in structure. The amphiphilic bilayer-forming agent can be phospholipid or a ceramide.

Multiple lipophilic side chain amphiphilic bilayer-forming substances are amphiphilic bilayer-forming substances have two or more lipophilic side chains (e.g., that are attached to a polar head group). Such lipids may be nonionic, cationic, anionic, zwitterionic in nature. Examples of suitable multiple lipophilic side chain amphiphilic bilayer-forming substances include, but are not limited to, those bilayer-forming cationic lipids that contain two saturated or unsaturated fatty acid chains (e.g., side chains having from about 10 to about 30 carbon atoms) such as di (soyoylethyl) hydroxyethylmonium methosulfate (DSHM), N-[I-(2,3-dioleyloxy)propyl]-N,N,N-trimethylammonium bromide (DOTMA), 1,2dimyristyloxypropyl-N,N-dimethyl-hydroxyethyl ammonium bromide (DMRIE), [N—(N,N′-dimethylaminoethane) carbamoyl] cholesterol (DC-Chol), dioctadecylamidoglycyl spermidine (DOGS), dimethyl dioctadecylammonium bromide (DDAB), dioleoyl phosphatidylethanolamine (DOPE), 2,3-dioleoyloxy]-N[2(sperminecarbozamide-O-ethyl]-N,N-dimethyl-propanaminium trifluoroacetate (DOSPA), I-[2-(oleoyloxy)-ethyl]-2-oleyl-3-(2hydroxyethyl) imidazolinium chloride (DOTIM), 1,2-dioleoyloxy-3-(trimethylammonio) propane (DOTAP), 1,2-diacyl-3-trimethylammonium propane (TAP), 1,2-diacyl-3-dimethylammonium propane (DAP), fatty acid salts of quaternary amines such as dicocodimonium chloride (Quaternium 34), and quaternary dimethyldiacyl amines wherein the acyl groups have from about 8 carbon atoms to about 30 carbon atoms (e.g., from about 10 carbon atoms to about 30 carbon atoms), and derivatives thereof such as ammonium derivatives, i.e. dimethyl dihydrogenated tallow ammonium chloride (Quaternium 18), and decyl dimethyl octyl ammonium chloride (Quaternium 24), and derivatives thereof. Other suitable cationic dual chain lipids are further described in the following references: Fasbender et al., 269 Am J Physiol L45-L5 1 (1995); Solodin et al., 34 Biochemistry 13537-13544 (1995); Felgner et al., 269 J Biol Chem 2550-2561(1994); Stamatatos et al., 27 Biochemistry 3917-3925 (1988); and Leventis and Silvius, 1023 Biochim Biophys Acta 124-132 (1990), and Jouani et al., 9 J. Liposome Research 95-114 (1999), which are all incorporated by reference herein.

Examples of suitable nonionic multiple lipophilic side chain amphiphilic bilayer-forming substances include, but are not limited to, glyceryl diesters, and alkoxylated amides. Examples of suitable glyceryl diesters include, but are not limited to, those glyceryl diesters having from about 10 carbon atoms to about 30 carbon atoms (e.g., from about 12 carbon atoms to about 20 carbon atoms), glyceryl dilaurate (“GDL”), glyceryl dioleate, glyceryl dimyristate, glyceryl distearate (“GDS”), glyceryl sesuioleate, glyceryl stearate lactate, and mixtures thereof, with glyceryl dilaurate, glyceryl distearate and glyceryl dimyristate, and derivatives thereof.

Examples of anionic multiple lipophilic side chain amphiphilic bilayer-forming substances include, but are not limited to, phosphatidic acids such as 1,2 dimyristoyl-sn-glycero-3-phosphate, sodium salt (DMPA), 1,2 dipalmitoyl-sn-glycero-3-phosphate, sodium salt (DPPA), 1,2 distearoyl-sn-glycero-3-phosphate, sodium salt (DSPA) and negatively charged phospholipids such as dipalmitoyl phosphatidylglycerol.

The amount of multiple lipophilic side chain amphiphilic bilayer-forming substances in the vesicle bilayer may range from, based upon the total weight of the substances in the lipid bilayer(s), from about 0.001 percent to about 95 percent (e.g., from about 5 percent to about 65 percent). The amount of multiple lipophilic side chain amphiphilic bilayer-forming substances based upon the total weight of the components in the liposome will depend upon the type of liposome (e.g., unilamellar or paucilamellar liposomes), and may range from about 0.001 percent to about 95 percent (e.g., from about 1 to about 65 percent).

A single lipophilic chain amphiphilic bilayer-forming substance is a amphililic bilayer forming substance containing a single lipophilic side chain (e.g., attached to a polar head group). The single chain lipids may be nonionic, cationic, anionic, or zwitterionic.

Examples of suitable nonionic single lipophilic chain amphiphilic bilayer-forming substances include, but are not limited to, glyceryl monoesters; polyoxyethylene fatty ethers wherein the polyoxyethylene head group has from about 2 to about 100 groups and the fatty acid tail group has from about 10 to about 26 carbon atoms; alkoxylated alcohols wherein the alkoxy group has from about 1 carbon atoms to about 200 carbon atoms and the fatty alkyl group has from about 8 carbon atom to about 30 carbon atoms (e.g., from about 10 carbon atoms to about 24 carbon atoms); alkoxylated alkyl phenyls wherein the alkoxy group has from about 1 carbon atoms to about 200 carbon atoms and the fatty alkyl group has from about 8 carbon atom to about 30 carbon atoms (e.g., from about 10 carbon atoms to about 24 carbon atoms); polyoxyethylene derivatives of polyol esters; alkoxylated acids wherein the alkoxy group has from about 1 carbon atoms to about 200 carbon atoms and the fatty alkyl group has from about 8 carbon atom to about 30 carbon atoms (e.g., from about 10 carbon atoms to about 24 carbon atoms); and alkoxylated acids.

Examples of suitable glyceryl monoester nonionic single lipophilic chain amphiphilic bilayer-forming substances include, but are not limited to, those glyceryl monoesters having from about 10 carbon atoms to about 30 carbon atoms (e.g., from about 12 carbon atoms to about 20 carbon atoms), glyceryl caprate, glyceryl caprylate, glyceryl cocoate, glyceryl erucate, glyceryl hydroxystearate, glyceryl isostearate, glyceryl lanolate, glyceryl laurate, glyceryl linolate, glyceryl myristate, glyceryl oleate, glyceryl PABA, glyceryl palmitate, glyceryl ricinoleate, glyceryl stearate, and glyceryl thiglycolate, and derivatives thereof.

Examples of suitable polyoxyethylene fatty ether nonionic single lipophilic chain amphiphilic bilayer-forming substance include, but are not limited to, polyoxyethylene cetyl ether, polyoxyethylene stearyl ether, polyoxyethylene cholesterol ether, polyoxyethylene laurate, polyoxyethylene dilaurate, polyoxyethylene stearate, polyoxyethylene distearate, polyoxyethylene lauryl ether, polyoxyethylene stearyl ether, polyoxyethylene myristyl ether, and polyoxyethylene lauryl ether, e.g., with each ether having from about 3 to about 10 oxyethylene units, and derivatives thereof.

Suitable examples of an alkoxylated alcohol nonionic single lipophilic chain amphiphilic bilayer-forming substance include, but are not limited to, those having the structure shown in formula I below:
R₅—(OCH₂CH₂)y—OH Formula I
wherein R₅ is an unbranched alkyl group having from about 10 to about 24 carbon atoms and y is an integer between about 4 and about 100 (e.g., from about 10 and about 100). An example of such an alkoxylated alcohol is the species wherein R₅ is a lauryl group and y has an average value of 23, which is known by the CTFA name “laureth 23” and is available from Uniqema, Inc. of Wilmington, Del. under the tradename BRIJ 35®.

Suitable examples of an alkoxylated alkyl phenyls nonionic single lipophilic chain amphiphilic bilayer-forming substance include, but are not limited to, those having the structure shown in formula II below: 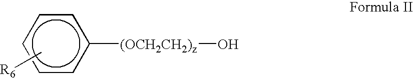
wherein R₆ is an unbranched alkyl group having from about 10 to about 24 carbon atoms and z is an integer of from about 7 and 120 (e.g., from about 10 to about 100). An example of this class of materials is the species wherein R₆ is a nonyl group and z has an average value of about 14. This material is known by the CTFA name “nonoxynol-14” and is available under the tradename, MAKON 14® from the Stepan Company of Northfield, Ill.

Suitable polyoxyethylene derivatives of polyol ester nonionic single lipophilic chain amphiphilic bilayer-forming substance are those wherein the polyoxyethylene derivative of polyol ester that: (1) is derived from (a) a fatty acid containing from about 8 to about 22 (e.g., from about 10 to about 14 carbon atoms) and (b) a polyol selected from sorbitol, sorbitan, glucose, α-methyl glucoside, polyglucose having an average of about 1 to about 3 glucose residues per molecule, glycerine, and pentaerythritol; (2) contains an average of from about 10 to about 120 (e.g., from about 20 to about 80) oxyethylene units; and (3) has an average of from about 1 to about 3 fatty acid residues per mole of polyoxyethylene derivative of polyol ester.

Examples of polyoxyethylene derivatives of polyol esters include, but are not limited to, PEG-80 sorbitan laurate and Polysorbate 20. PEG-80 sorbitan laurate, which is a sorbitan monoester of lauric acid ethoxylated with an average of about 80 moles of ethylene oxide, is available commercially from ICI Surfactants of Wilmington, Del. under the tradename Atlas G4280®. Polysorbate 20, which is the laurate monoester of a mixture of sorbitol and sorbitol anhydrides condensed with approximately 20 moles of ethylene oxide, is available commercially from ICI Surfactants of Wilmington, Del. under the tradename Tween 20®. Another exemplary polyol ester is sorbitan stearate, which is available from Uniqema, Inc. under the tradename SPAN 60®.

Suitable examples of alkoxylated acid nonionic single lipophilic chain amphiphilic bilayer-forming substance include, but are not limited to, the esters of an acid (e.g., a fatty acid) with a polyalkylene glycol. An exemplary material of this class has the CTFA name PEG-8 laurate®.

Examples of suitable cationic single lipophilic chain amphiphilic bilayer-forming substance include, but are not limited to, quaternary trimethylmonoacyl amines wherein the acyl groups have from about 8 carbon atoms to about 30 carbon atoms (e.g., from about 10 carbon atoms to about 24 carbon atoms), and derivatives thereof such as ammonium derivatives, e.g., stearamidopropyl dimethyl (myristyl acetate) ammonium chloride (Quaternium 70), triethyl hydrogenated tallow ammonium chloride (Quaternium 16), and benzalkonium chloride, and derivatives thereof.

Examples of suitable anionic single lipophilic chain amphiphilic bilayer-forming substances include, but are not limited to, fatty acids such as oleic acid and negatively charged single chained phospholipids such as phosphatidylserine and phosphatidylglycerol.

The amount of single lipophilic chain amphiphilic bilayer-forming substance in the vesicle bilayer may range from, based upon the total weight of the substances in the lipid bilayer(s), from about 0.001 percent to about 70 percent (e.g., from about 1 percent to about 30 percent). The amount of single lipophilic chain amphiphilic bilayer-forming substance based upon the total weight of the components in the liposome will depend upon the type of liposome (e.g., unilamellar or paucilamellar liposomes), and may range from about 1 percent to about 95 percent (e.g., from about 1 percent to about 30 percent).

The above single and multiple lipophilic chain amphiphilic bilayer-forming substance may also be a phospholipid, which may be zwitterionic in nature. Examples of phospholipids include, but are not limited to, natural and synthetic phospholipids. Examples of natural phospholipids include, but are not limited to, egg phosphatidylcholine, hydrogenated egg phosphatidylcholine, soybean derived phospholipids such as soybean phosphatidylcholine, phospholipids from plant sources, sphingomyelin. Examples of synthetic phospholipids include, but are not limited to, synthetic phosphatidylcholines such as 1,2-dilauroyl-sn-glycero-3-phosphocholine (DLPC), 1,2-dimyristoyl-sn-glycero-3-phosphocholine (DMPC), 1,2-dipalmitoyl-sn-glycero-3-phosphocholine (DPPC), 1,2-distearoyl-sn-glycero-3-phoshpocholine(DSPC), 1,2-didioleoyl-sn-glycero-3-phoshpocholine(DOPC), 1-palmitoyl-2-oleoyl-sn-glycero-3-phoshpocholine(POPC), phosphatidylethanolamines include, but are not limited to, 1,2-dimyristoyl-sn-glycero-3-phoshpethanolamine(DMPE), 1,2-dipalmitoyl-sn-glycero-3-phoshpethanolamine(DPPE), 1,2-distearoyl-sn-glycero-3-phoshpethanolamine(DSPE), 1,2-dioleoyl-sn-glycero-3-phoshpethanolamine(DOPE), negatively charges phospholipids such as dipalmitoyl phosphatidylglycerol (DPPG), dipalmitoyl phosphatidylcholine (DPPC), dipalmitoyl phosphatidic acid (DPPA), and phosphatidylserine (PS), and derivatives thereof.

The above single and multiple lipophilic chain amphiphilic bilayer-forming substance may also be a cermide. Examples include, but are not limited to, N-acetyl D-erythro-sphinogsine(C2Cer), N-octanoyl D-erythro-sphinogsine (C8Cer), N-myristoyl D-erythro-sphinogsine(C14Cer), N-stearoyl D-erythro-sphinogsine(C18Cer), N-arachidoyl D-erythro-sphinogsine(C20Cer).

Other suitable lipids are further described in the following references: Avanti Polar Lipids, Inc., Alabaster, Ala. Interim Catalog 13-92 and 105-127 (1999); polyglycerol such as those described in U.S. Pat. No. 4,772,471, French Patent Nos. 1,477,048 and 2,091,516;; amide-based oligomeric cationic lipids such as those described in U.S. Pat. No. 5,877,220; cationic lipids such as those described in U.S. Pat. Nos. 5,980,935, 5,851,548, 5,830,430, and 5,777,153; phosphonic acid-based cationic lipids such as those described in U.S. Pat. No. 5,958,901; quaternary cytofectins such as those described in U.S. Pat. No. 5,994,317; ether lipids such as those described in U.S. Pat. No. 5,989,587; and polyethylene glycol modified cermide lipids such as those described in U.S. Pat. No. 5,820,873.

Sterols may be added to the lipid bilayer of the liposome. The presence of a rigid steroid alongside the fatty acid chains of the lipid in the bilayer may reduce the freedom of motion of these carbon chains, creating better packing of the lipid bilayers. Examples of suitable sterols include, but are not limited to, cholesterol and salts and esters thereof, cholesterol 3-sulfate, phytocholesterol, hydrocortisone, alpha-tocopherol, betasitosterol, bisabolol and derivatives thereof.

The amount of sterol in the vesicle bilayer may range from, based upon the total weight of the substances in the vesicle bilayer, from about 0.001 percent to about 95 percent (e.g., from about 1 percent to about 65 percent). The amount of sterol, based upon the total weight of the components in the liposome will depend upon the type of liposome (e.g., unilamellar or paucilamellar liposomes), and may range from about 0.001 percent to about 95 percent (e.g., from about 1 percent to about 65 percent).

The benefit agent may be contained within the lipid bilayer (e.g., if it is a lipophilic agent) or within a hydrophilic component of the liposome (e.g., within the hydrophilic regions within the lipid bilayers of within the core). The hydrophilic component may contain water and/or other polar solvents. Examples of polar solvents include, but are not limited to, glycols such as glycerin, alcohols (e.g., those alcohols having from about 2 carbon atoms to about 6 carbon atoms), propylene glycol, sorbitol, oxyalkylene polymers such as PEG 4, and derivatives thereof.

The liposomes of the present invention may be included within pharmaceutical (e.g., compounded with a pharmaceutically compatible carrier). The resulting composition may be in the form of a cream, ointment, lotion, gel for therapeutic use.

It is also envisaged that the liposomal formulation of the present invention may include additional benefit agents for the treatment of osteoarthritis, including analgesics, anti-inflammatory agents, or chondroprotective agents. Typical examples of such benefit agents include, but are not limited to, non-steroidal anti-inflammatory drugs, p38 kinase inhibitors, TNF-α inhibitors, corticosteroids, inhibitors of enzymes that are involved in the destruction of articulating joints or synovial fluid components, such as hyaluronidase inhibitors, matrix metalloproteinase inhibitors, or aggrecanase inhibitors, apoptosis inhibiors such as EPO, and cartilage enhancing factors such as TGF-β1, and Bone Morphogenetic Proteins. The extra benefit agent thus described may be either co-encapsulated with the GAG, bound to the liposome but not encapsulated, or present as free drug outside the liposome bilayers.

The following is a description of the manufacture and testing of liposomes of the present invention. Other liposomes of the invention can be prepared in an analogous manner by a person of ordinary skill in the art. The desciption of formulation methodology outlined below is considered only as an example, and it is understood that other methods of producing formulations encapsulating GAGs in liposomes may also be effective. Examples of such methods are described in detail by Vemuri and Rhodes (1995) and include, but are not limited to, mixing of SUV with aqueous phase containing the benefit agent, subsequent lyophilisation and rehydration to yield MLV [e.g. Kirby and Gregoriadis, 1984], reverse-phase evaporation [Szoka and Papahadjopoulos, 1978], high pressure extrusion [Vemuri et. al, 1990], freeze-thaw of liposomes [Pick, 1981] and dehydration/rehydration [Shew and Deamer, 1985].

### EXAMPLE 1

A. Preparation of Hyaluronic Acid

HA may be prepared by any method. However, the preferred method would be to produce HA to a high purity through a bacterial fermentation route such as that described in WO86/04355.

B. Preparation of Liposomes with Encapsulated Hyaluronic Acid

Hyaluronic acid was incorporated into 1,2-dipalmitoyl-sn-glycero-3-phosphocholine (DPPC) liposomes was carried out by a film hydration method. Briefly, DPPC (400 mg) was dissolved in 40 ml of ethanol. The DPPC/ethanol mixture was then placed in a round bottom flask and attached to a rotary evaporator apparatus and then the round bottom flask was lowered into a water bath (Buchi Labortechnik AG, Switerland). At a rotation speed of 54 rpm, the ethanol in the mixture was slowly removed by vacuum and the resulting film was stored under vacuum for 1 hour. The thin film was then hydrated with 4 ml of a phosphate buffer saline (PBS) solution at 55° C. After the lipid film was hydrated, 4 ml of 20 mg/ml hyaluronic acid solution was added to the lipid/PBS mixture and was then vortexed in 15 second interval for 5 minutes at 55° C. The newly formed vesicles were allowed to equilibrium ambient condition before testing. The final liposomal concentration was 50 mg/ml DPPC and 10 mg/ml hyaluronic acid.

C. Preparation of Blank Liposomes

Blank liposomes (without hyaluronic acid) were prepared using the same method above but without the addition of HA. The final liposomal concentration was 100 mg/ml DPPC.

D. Preparation of Liposomes with Non-Encapsulated Hyaluronic Acid

To prepare liposomal mixture where the hyaluronic acid was not encapsulated, the 4 ml of the blank liposomes of Example 2 were mixed with 4 ml of 20 mg/ml hyaluronic acid solution. The final liposomal concentration was 50 mg/ml DPPC and 10 mg/ml hyaluronic acid.

E. Freeze Fracture Microscopy

The compositions were subsequently examined using a freeze-fracture transmission electron microscope (FF-TEM). FF-TEM samples of each formulation were prepared in accordance with techniques described in chapter 5 of “Low Temperature Microscopy and Analysis” by Patrick Echlin (1992). The sample was mounted between thin metal sheets and rapidly cooled with liquid propane to −196° C. The sample was then transferred under liquid nitrogen to a pre-cooled cold stage of a Balzers BAF-301 high vacuum freeze-etch unit (Techno Trade International, Lichtenstein). The sample was fractured at low temperature and etched at −150° C. to remove a surface layer of water. The fracture faces were shadowed at an angle of 45° with platinum to create selective electron contrast. A thin layer of carbon was deposited over the entire fracture surface to create a continuous replica. The replicas were then examined using a JEOL 100CX2 electron microscope (Japanese Electronic Optical Laboratories, Japan).

Hyaluronic acid solution showed the presence of string like structures.

Liposomes of Example 2 showed the presence of intact vesicles with bilayers. There was no evident on hyaluronic acid in the external phase. All the hyaluronic acid appeared to be encapsulated inside the DPPC liposomes.

Liposomal mixture showed liposomal structures and string like structures indicating the presence of hyaluronic acid not encapsulated inside the DPPC liposomes.

### REFERENCES

The following publications and patents are herein incorporated by reference:
Adams, M. E. An analysis of clinical studies of the use of crosslinked hyaluronan, Hylan, in the treatment of osteoarthritis. J. Rheumatol. 20 (Suppl. 39):16-18, 1993. Adams, M. E., Atkinson, M. H., Lussier, A., Schulz, J1, Siminovitch, K. A., Wade, J. P., and Zummer, M. (1995). The role of viscosupplementation with Hylan G-F 20 (Synvisc(g) in the treatment of osteoarthritis of the knee. Osteoarthritis and Cart. 3:213-226, 1995.
Adams, M. E. Viscosupplementation as articular therapy. In The Chemistry, Biology and Medical Applications of hyaluronan and its derivatives (Proceedings of Wenner-Gren Foundation International Symposium held in honor of Endre A. Balazs, Sep. 18-21, 1996 Stockholm, Sweden) (Ed. Laurent T., Portland Press, London), 243-253.
Baker, D. E. Pharmacy and Therapeutics Review. The Formulary, December 1997.
Balazs, E. A. Viscoelastic properties of hyaluronic acid and biological lubrication. Univ. Michigan. Med. Ctr. Journal, 255-259, 1968.
Balazs, E. A. and Gibbs, D. A. The Theological properties and biological function of hyaluronic acid. In Chemistry and Molecular Biology of the Intercellular Matrix (Ed. Balazs, E. A), Academic Press, London and New York, 1241-1254, 1970. Balazs, E. A. The physical properties of synovial fluid and the special role of hyaluronic acid. In Disorders of the Knee, Second Edition, (Ed. Heflet, A.), J. B. Lippincott Company, Philadelphia, 61-74, 1982.
Balazs, E. A. and Denlinger, J. L. Sodium hyaluronate and joint function. J. Equine Vet. Sci. 5:217-228, 1985.
Balazs, E. A. and Denlinger, J. L. Clinical uses of hyaluronan. In the Biology of Hyaluronan (Ciba Foundation Symposium #143) (eds. Evered, D. and Whelan, J.). John Wiley & Sons, Chichester and New York, 265-280, 1989.
Balazs, E. A. and Denlinger, J. L. Viscosupplementation: A new concept in the treatment of osteoarthritis. J. Rheumatol. 20 (Suppl. 39):3-9, 1993.
Band, P., Goldman, A., Barbone, A., Reiner, K., and Balazs, E. A. Intra-articular distribution and residence time of Hylan polymers. Materials Research Society, Spring Meeting, Apr. 17-21, 1995, San Francisco, Calif. 433 (abstract).
Bangham, A. D., Standish, M. M. and Watkins, J. C. 1965. “Diffusion of Univalent Ions across the lamellae of swollen phospholipid,” J. Mol. Biol., 13: 238-252). Denlinger. J. L. Metabolism of sodium hyaluronate in articular and ocular tissues. Ph.D. thesis, Universit6 des Sciences et Techniques de Lille, Lille, France, 1982. Denlinger, J. L. Hyaluronan and its derivatives as viscoelastics in medicine. In The Chemistry, Biology and Medical Applications of Hyaluronan and its Derivatives (Proceedings of the Wener-Gren Foundation International Symposium held in honor of Endre A. Balazs, Sep. 18-21, 1996, Stockholm, Sweden) (Ed. Laurent, T.), Portland Press, London, 235-242.
Dickson, D. J. and Hosle, G. Double-blind, double-control comparison of viscosupplementation with Hylan G-F 20 (Synvisc(R)) against dicolfenac and control in knee osteoarthritis. American College of Rheumatology, 62 d National Scientific Meeting, Nov. 8-12, 1998, San Diego. CA. Arthritis Rheum. 41 (9), S 197 (abstract).
Estey, D. Viscosupplementation—A new treatment for patients with osteoarthritis. Surgical Physician Assistant, 19-23), 1998.
Furuya, T.; Yamagata, S.; Shimoyama, Y.; Fujihara, M.; Morishima, N.; Ohtsuki, K. Biochemical characterization of glycyrrhizin as an effective inhibitor for hyaluronidases from bovine testis. Biological & Pharmaceutical Bulletin 20, 9, 973-977,1997.
Gibbs, D. A., Merrill, E. W., Smith, K. A., and Balazs, E. A. The theology of hyaluronic acid. Biopolymers, 6: 777-791, 1968.
Gregoriadis, G. 1988. liposomes as drug carriers: Recent trends and progress. Cchichester, UK: John Wiley & Sons.
Gregoriadis, G. 1992. Lipsoma technology, vols 1-3. 2^{nd} ed. Boca Raton, Fla. USA: CRC Press Inc.
Matveikov, G. P.; Soroka, N. F.; Maksimovich, A. B. The possibilities of an individual approach to intra-articular drug administration in rheumatoid arthritis patients Ter Arkh 61, 12, 121-4,1989.
Moreland, L. W., Arnold, W. J., Saway, A., Savory, C. and Sikes, D. Efficacy and safety of intra-articular Hylan G-F 20 (Synvisc(E), a viscoelastic derivative of hyaluronan, in patients with osteoarthritis of the knee. American College of Rheumatology, 57 Ih Annual Scientific Meeting, San Antonio, Tex., Nov. 7-11, 1993, (abstract).
Peyron, J. G. A new approach to the treatment of osteoarthritis: viscosupplementation. Osteoarthritis Cart. 1:85-87, 1993a.
Peyron, J. G. Intra-articular hyaluronan injections in the treatment of osteoarthritis: state-of-the-art review. J. Rheumatol. 20 (Suppl. 39): 10-15, 1993b.
Peyron, J. G. Viscosupplementation for the treatment of osteoarthritis of the knee with hyaluronan and Hylans: rationale and state of the art. In Advances in Osteoarthritis (Eds. Tanaka, S and Hamanishi, C.), Springer-Verlag, Tokyo, 213-236,1999.
Rydell, N. W. Butler. J., and Balazs, E. A. Hyaluronic acid in synovial fluid. VI. Effect of intra-articular injection of hyaluronic acid on the clinical symptoms of arthritis in track horses. Acta. Vet. Scand. 11: 139-155, 1970
Scale, D., Wobig, M., and Wolpert, W. Visco supplementation of osteoarthritic knees with Hylan: a treatment schedule study. Curr. Therap. Res. 55:220-232, 1994.
Weiss, C., Balazs, E. A., St Onge, R. and Denlinger, J. L. Clinical studies of the intra-articular injection of Healon®R (sodium hyaluronate) in the treatment of osteoarthritis of human knees. In Seminars in Arthritis and Rheumatism, Vol 11, (Ed. Talbott, J. H.) Grune and Stratton, New York, 1431-144, 198 1.
Weiss, C. and Balazs, E. A. Arthroscopic viscosurgery. Arthroscopy 3:138-139, 1987.
Peyron, J. G. and Balazs, E. A., Preliminary Clinical Assessment of Na-Hyaluronate Injection into Human Arthritic Joints. Path. Biol., 1974, 22 (8), 731-6 Adams, M. E., An Analysis of Clinical Studies of the Use of Crosslinked Hyaluronan, Hylan, in the Treatment of Osteoarthritis. Journal of Rheumatology, 1993, 20 (S39), 16-18
Adams, M. E., Atkinson, M. H., Lussier, A. J., Schulz, J. I., Siminovitch, K. A., Wade, J. P. and Zummer, M., The Role of Viscosupplementation with Hylan G-F 20 (Synvisc®) in the Treatment of Osteoarthritis of the Knee: a Canadian Multicenter Trial Comparing Hylan G-F 20 Alone, Hylan G-F 20 with Non-Steroidal Anti-Inflammatory Drugs (NSAIDs) and NSAIDS Alone. Osteoarthritis and Cartilage, 1995, 3, 213-226.
Huskisson, E. C. and Donnelly, S., Hyaluronic Acid in the Treatment of Osteoarthritis of the Knee. Rheumatology, 1999, 38, 602-607
Kotz, R. and Kolarz, G., Intra-articular Hyaluronic Acid: Duration of Effect and Results of Repeated Treatment Cycles. American Journal of Orthopaedics, 1999, 28 (11), 5-7
Balazs, E. A. and Denlinger, J. L—‘Sodium hyaluronate and joint function’. Eq. Vet. Sci., 1984, 5, pp. 217-228
Balazs, E. A., and Darzynkiewicz, Z. In: Kulonen, E and Pikkarainen, J, eds. Biology of the Fibroblast. London: Academic Press, 1973: 237-52
Forrester, J. V., and Balazs, E. A., Immunology 1980; 40:435-46
Darzynkiewicz, Z., and Balazs, E. A., Exp Cell Res 1971; 66:113-23
FDA-CDRH-PMA 950027 Hyalgan, 1997a.
FDA-CDRH-PMA 940015 Hylan G-F 20, 1997b.
Levick J R, Scott D, Coleman P J, Mason R M, ‘Role of hyaluronan chain length in buffering interstitial flow across the synovium in rabbits’ J of Physiology, 526.2, pp. 425-434, (2000)
Hills, B. A., Boundary lubrication in vivo. Proc Inst Mech Eng [H]. 2000;214(1): 83-94.
Hills, B. A., Remarkable anti-wear properties of joint surfactant. Annals of Biomedical Engineering. 1995, 23, 112-115.
Hills, B. A. and Monds, M. K., Enzymatic identification of the load-bearing boundary lubricant in the joint. British Journal of Rheumatology, 1998, 37, 137-142.
Vecchio, P, Thomas, R and Hills, B. A. Surfactant treatment for osteoarthritis. Rheumatology (Oxford). 1999 October;38(10):1020-1
Moro-oka T, Miura H, Mawatari T, Kawano T, Nakanishi Y, Higaki H, Iwamoto Y, Mixture of hyaluronic acid and phospholipid prevents adhesion formation on the injured flexor tendon in rabbits. J Orthop Res 2000, 18(5): 835-40
Pasquali-Ronchetti, I. et al, Hyaluronan-Phospholipid Interactions, Journal of Structural Biology, 1997, 120, 1-10
Ghosh, P., Hutadilok, N., Adam, N. and Lentini, A., Interactions of hyaluronan (hyaluronic acid) with phospholipids as determined by gel permeation chromatography, multi-angle laser-light-scattering photometry and ¹H-NMR spectroscopy. Int. J. Biol. Macromol. 1994, 16 (5), 237-244.
Bangham, A. D., Standish, M. M. and Watkins, J. C. 1965. “Diffusion of Univalent Ions across the lamellae of swollen phospholipid,” J. Mol. Biol., 13: 238-252
Callow and McGrath, Cryobiology, 1985 22(3) pp. 251-267
Gregoriadis, G., ed. 1988. Liposomes as Drug Carriers, New York: John Wiley, pp. 3-18
Fasbender et al., 269 Am J Physiol L45-L5 1 (1995)
Solodin et al., 34 Biochemistry 13537-13544 (1995)
Felgner et al., 269 J Biol Chem 2550-2561(1994)
Stamatatos et al., 27 Biochemistry 3917-3925 (1988)
Leventis and Silvius, 1023 Biochim Biophys Acta 124-132 (1990)
Jouani et al., 9 J. Liposome Research 95-114 (1999)
Vemuri, S. and Rhodes, C. T., Pharmaceutica Acta Helvetiae, 70, 95-111 (1995)
Kirby, C. J., and Gregoriadis, G., A simple procedure for preparing liposomes capable of high encapsulation efficiency under mild conditions. In: Liposome Technology, Vol. 1, CRC Press, Boca Raton, Fla., 1984
Szoka and Papahadjopoulos, Biochem., 1978, 75, 4194-8
Vemuri, S. et. al, Dug Dev. Ind. Pharm., 1990, 16, 2243-2256
Pick, U. I., Arch. Biochem. Biophys., 1981, 212, 1986-1999
Shew, R. L., and Deamer, D., Biochim Biophys Acta, 1985, 816, 1-8

1. A composition useful for the treatment of arthritic joints comprising at least one glycosaminoglycan, at least part of which are encapsulated in at least one liposome.

2. The composition of claim 1, in which the glycosaminoglycan is selected from the group consisting of: chondroitin sulphate, keratinsulphate, heparin, heparin sulphate and dermatan sulphate.

3. The composition of claim 1, wherein the glycosaminoglycan is hyaluronic acid.

4. The Composition of claim 1, wherein the glycosaminoglycan is of greater than 500 kDa molecular weight.

5. The Composition of claim 1, wherein at least 10% by volume of the glycosaminoglycan is encapsulated in at least one liposome.

6. The composition of claim 1, wherein the concentration of glycosaminoglycan is greater than 1 mg/ml.

7. The composition of claim 1, wherein the liposome is made from a bilayer-forming phospholipid.

8. The composition of claim 8, wherein the phospholipid is dipalmitoylphosphatidylcholine.

9. The composition of claim 1, which additionally includes a further benefit agent for the treatment of osteoarthritis

10. The composition of claim 1, wherein the lipid concentration is greater than 10 mg/ml.

11. A liposomal delivery vehicle which encapsulates one or more glycosaminoglycans.

12. The liposomal delivery vehicle of claim 11, which is spherical or rod-shaped in structure.

13. The liposomal delivery system of claim 11 which has a diameter of greater than 0.1 μm.

14. A method for the treatment of arthritic joints, The method comprising the steps of: a) preparing the composition of claim 1 and b) administering the composition in a pharmaceutically appropriate dosage.

15. The method of claim 14, wherein the condition treated is osteoarthritis.

16. A method of claim 14, wherein the method of administration is by intra-articular injection of the composition into the arthritic joint.
